(19) United States
(12) Patent Application Publication
(10) Pub. No.: US 2003/0236246 A1
(43) Pub. Date: Dec. 25, 2003

(54) Method for decreasing capillary permeability in the retina

(76) Inventors: Romulus Kimbro Brazzell, Alpharetta, GA (US); Kenneth E. Green, Alpharetta, GA (US); Frances Elizabeth Kane, Cumming, GA (US); Peter Anthony Campochiaro, Baltimore, MD (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/419,410

(22) Filed: Apr. 21, 2003

Related U.S. Application Data
(60) Provisional application No. 60/376,831, filed on Apr. 30, 2002.

(51) Int. Cl. A61K 31/553

(52) U.S. Cl. 514/211.08

(57) ABSTRACT

Disclosed are methods for decreasing or attenuating an increase in capillary permeability in the retina in a subject in need of such treatment, comprising administering a composition comprising an amount of a staurosporine derivative or a salt thereof to a subject suffering from excessive or pathological capillary permeability in the retina, the amount of staurosporine derivative or salt thereof being effective to decrease the permeability of capillaries in the retina of the subject.

The blood-retinal barrier (BRB) consists of an inner component, comprised of the retinal capillary endothelial cells, and an outer component, comprised of retinal pigment epithelial cells. The BRB separates the retina from the efflux of the fenestrated vessels, primarily capillaries, of the choroid. The BRB is established by complexly arranged tight junctions between the barrier-forming cells and a paucity of endocytic vesicles within these cells. Both components of the BRB function by excluding blood-borne proteins and small, water-soluble non-electrolytes from the retina and by maintaining cell polarity through ionic and metabolic gradients. Increased permeability of the BRB and subsequent leakage can lead to diabetic macular edema, the primary cause of loss of vision in early diabetic retinopathy.

In one aspect, the present invention relates to a method for decreasing capillary permeability in the retina in a subject in need of such treatment, comprising administering a composition comprising an amount of a staurosporine derivative or a salt thereof to a subject suffering from excessive or pathological capillary permeability in the retina, the amount of staurosporine derivative or salt thereof being effective to decrease the permeability of capillaries in the retina of the subject.

In a further aspect, the invention relates to a method for decreasing capillary permeability in the retina in a subject in need of such treatment, comprising administering a composition comprising an amount of a staurosporine derivative or a salt thereof to a subject suffering from excessive or pathological capillary permeability in the retina, the amount of staurosporine derivative or salt thereof being effective to decrease the permeability of capillaries in the retina of the subject, where the subject is suffering from clinically significant diabetic macular edema.

In a further aspect, the invention relates to a method for decreasing capillary permeability in the retina in a subject in need of such treatment, comprising administering a composition comprising an amount of a staurosporine derivative or a salt thereof to a subject suffering from excessive or pathological capillary permeability in the retina, the amount of staurosporine derivative or salt thereof being effective to decrease the permeability of capillaries in the retina of the subject, where the subject is not suffering from clinically significant macular edema.

In a further aspect, the invention relates to a method for decreasing capillary permeability in the retina in a subject in need of such treatment, comprising administering a composition comprising an amount of a staurosporine derivative or a salt thereof to a subject suffering from excessive or pathological capillary permeability in the retina, the amount of staurosporine derivative or salt thereof being effective to decrease the permeability of capillaries in the retina of the subject, where the subject is suffering from visual acuity loss.

In another aspect, the invention relates to the practice of the abovementioned methods where the effective amount is less than about 150 milligrams per day by oral administration.

In another aspect, the invention relates to the practice of the abovementioned methods where the effective amount is between about 50 and about 150 milligrams per day by oral administration.

In another aspect, the invention relates to the practice of the abovementioned methods where the total effective amount is delivered to the subject in a single administration at a single time point during the day.

In another aspect, the invention relates to the practice of the abovementioned methods where the total effective amount is delivered to the subject in multiple administrations at multiple time points during the day.

In another aspect, the invention relates to the practice of the abovementioned methods where the effective amount is administered topically to the eye.

In another aspect, the invention relates to the practice of the abovementioned methods where the effective amount is administered intravitreally, subconjunctivally, or peribulbarly to the eye.

In another aspect, the invention relates methods for treating a subject suffering from diabetic nephropathy or diabetic neuropathy comprising administering an effective amount of a composition comprising an amount of a staurosporine derivative or a salt thereof to a subject suffering from diabetic nephropathy or diabetic neuropathy.

As used herein, the term “clinically significant macular edema” means that a subject is suffering from one or more of (1) retinal edema within 500 micrometers from the center of the fovea (2) hard exudates within 500 micrometers of the fovea, if associated with adjacent retinal thickening, which thickening may be outside the 500 micrometer limit or (3) retinal edema that is within one disc area, i.e., 1500 micrometers, or larger any part of which is within one disc diameter of the center of the fovea.

As used herein, a subject in need of treatment who is not suffering from clinically significant macular edema is a subject who is suffering from an increase in capillary permeability in the retina where the increase in capillary permeability is detectable using methods familiar to those of ordinary skill in the art, including ophthalmoscopy and fluorescein angiography.

As used herein, the term “visual acuity loss” means reduction in the clarity of central vision due to increased retinal capillary permeability, such as that found in subjects diagnosed with diabetic macular edema.

There is provided in accordance with the present invention a method for decreasing capillary permeability in the retina of a subject in need of such treatment. The method comprises the step of administering an amount of a staurosporine derivative or a salt thereof to a subject suffering from excessive or pathological capillary permeability in the retina, more specifically capillary permeability in the retina structures that comprise the BRB, effective to decrease the capillary permeability.

The methods of the present invention use a composition containing a staurosporine derivative. It has now surprisingly been found that compounds disclosed in U.S. Pat. No. 5,093,330 are useful for decreasing capillary permeability in the retina.

The pharmaceutical composition of the present invention which contains a compound disclosed in U.S. Pat. No. 5,093,330 as the active ingredient can be administered enterally, nasally, buccally, rectally, topically, orally, and parenterally, e.g., intravenous, intramuscular, intravitreal, subconjunctival or subcutaneous administration, to decrease capillary permeability in mammalian subjects, especially humans. The compositions may contain the active ingredient alone or, preferably, the active ingredient along with a pharmaceutically acceptable carrier. The effective dosage of the active ingredient depends on the type of targeted disease, as well as the species, age, weight and physical condition of the subject, pharmacokinetic data, and the mode of administration. Examples of effective oral daily doses in humans are, e.g., between about 1 and about 250 milligrams, between about 10 and about 150 milligrams, between about 12.5 and about 150 milligrams, between about 25 and about 150 milligrams, between about 50 and about 150 milligrams, and between about 100 and about 150 milligrams. Dosages can be repeated on a daily basis as necessary to achieve the desired decrease in capillary permeability. Daily doses can be administered at one time point, or can be divided with the total daily dose being administered over several time points during the day.

When administered topically or intravitreally, effective dosage are between about 1 and about 250 milligrams, e.g., between about 10 and about 150 milligrams, between about 25 and 50 milligrams, or about 25 milligrams per administration.

The compounds useful in the methods of the invention are administered in an amount effective to decrease capillary permeability in the retina of a subject suffering from excessive or pathological capillary permeability in the retina. Suitable pharmaceutical compositions may have from about 1% to about 95% of the active ingredient. Suitable unit dose forms include coated and uncoated tablets, ampoules, vials, suppositories, or capsules. Other suitable dosage forms include injectables, intraocular devices, intravitreal devices, ointments, creams, pastes, foams, tinctures, eye-drops, oral drops, sprays, dispersions and the like. The pharmaceutical compositions useful in the methods of the present invention are prepared in a manner known in the art, for example, by means of conventional mixing, granulating, coating, dissolving or lyophilizing processes.

Solutions of the active ingredient, and also suspensions or dispersions, especially isotonic aqueous solutions, dispersions or suspensions are also useful in the practice of the invention. Suitable useful pharmaceutical compositions containing the active ingredient may have carriers, e.g., mannitol and starch, preservatives, stabilizers, wetting agents, emulsifiers, solubilizers, salts for regulating osmotic pressure, buffers and the like. The compositions are prepared in a manner known in the art, for example by means of conventional dissolving and lyophilizing processes. A solution or suspension form of the composition may contain viscosity-increasing agents, e.g., sodium carboxymethylcellulose, carboxymethylcellulose, dextran, polyvinylpyrrolidone, and gelatins; and solubilizers, e.g., Tween 80 (polyoxyethylene(20)sorbitan mono-oleate; trademark of ICI Americas, Inc, USA).

Suitable carriers include fillers, e.g., sugars, for example lactose, saccharose, mannitol or sorbitol; cellulose preparations; calcium phosphates, e.g., tricalcium phosphate and calcium hydrogen phosphate; binders, e.g., starches, methylcellulose, hydroxypropyl methylcellulose, sodium carboxymethylcellulose and polyvinylpyrrolidone; and, if desired, disintegrators, e.g., starches, crosslinked polyvinylpyrrolidone, alginic acid or salts thereof. Additional suitable excipients are flow conditioners and lubricants, e.g., silicic acid, talc, stearic acid and salts thereof, such as magnesium or calcium stearate, polyethylene glycol, and derivatives thereof.

In addition to the active ingredients, pharmaceutical compositions useful in the practice of the presently claimed methods may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

The present invention also provides for administration of a pharmaceutical composition comprising a solution or dispersion of a staurosporine active ingredient in a saturated polyalkylene glycol glyceride.

The kinase inhibitor active ingredients may be any of the staurosporine derivatives described in U.S. Pat. No. 5,093,330. Preferred compounds are N-acylstaurosporines including N-benzoyl staurosporine, N-(3-nitrobenzoyl)staurosporine, N-(3-fluorobenzoyl)staurosporine, N-trifluoracetylstaurosporine, N-phenylcarbamoylstaurosporine, N-(3-carboxypropionyl)staurosporine, N-methylaminothiocarbonylstaurosporine, N-tert-butoxycarbonylstaurosporine, N-(4-carboxybenzoyl)staurosporine, N-(3,5-dinitrobenzoyl)staurosporine, N-(2-aminoacetyl)staurosporine, N-alanylstaurosporine and their pharmaceutically acceptable salts. An especially preferred active ingredient is N-benzoyl staurosporine.

The saturated polyalkylene glycol glyceride may be, for example, a mixture of glyceryl and polyethylene glycol esters of one or more long chain saturated fatty acids, usually C₈-C₁₈ saturated fatty acids. The acid component of such esters may be, for example, caprylic acid, capric acid, lauric acid, myristic acid, palmitic acid, stearic acid or a mixture of two or more thereof. The polyethylene glycol component of such esters generally has a molecular weight of 200 to 2000, preferably 1000 to 1800, especially 1400 to 1600. The glycerides, i.e. the glycol-modified glycerides, are usually mixtures of mono, di and triglycerides and polyethylene glycol mono and diesters.

Preferred polyalkylene glycol glycerides are those having a high Hydrophilic-Lipophilic Balance (HLB) value. Further preferred are glycerides which are mixtures of esters of one or more C₈-C₁₈ saturated fatty acids with glycerol and a polyethylene glycol having a molecular weight of 1000 to 2000, preferably 1200 to 1800, especially 1400 to 1600. An especially preferred material is available commercially from Gattefosse as Gelucire 44/14; this is a mixture of esters of C₈-C₁₈ saturated fatty acids with glycerol and a polyethylene glycol having a molecular weight of about 1500, the specifications for the composition of the fatty acid component being, by weight, 4-10% caprylic acid, 3-9% capric acid, 40-50% lauric acid, 14-24% myristic acid, 4-14% palmitic acid and 5-15% stearic acid.

The saturated polyalkylene glycol glycerides are either commercially available or may be prepared by known procedures. For example they may be obtained by partial alcoholysis of hydrogenated vegetable oils using the polyalkylene glycol, or by esterification of the saturated fatty acid, or mixture of such acids, using glycerol and the polyalkylene glycol.

In compositions of the invention, the kinase inhibitor active ingredient is generally present in an amount from 1 to 30%, preferably 5 to 25%, especially 10 to 20%, by weight of the composition.

The compositions of the invention may also contain carriers or adjuncts such as those described in U.S. Pat. No. 5,093,330 or other conventional excipients. For oral administration, the composition may be contained in capsules, usually hard capsules of gelatin or soft capsules of gelatin mixed with a plasticizer such as glycerol or sorbitol, or may be used as a dispersion in an aqueous medium, such as water, saline solution or a mixture of water with another, water-miscible, pharmaceutically acceptable solvent, for example in an amount of 0.5 to 70, preferably 5 to 50% by weight, optionally together with a preservative, for example a conventional preservative such as a benzoate, particularly an ester of p-hydroxybenzoic acid such as the methyl, ethyl, n-propyl, n-butyl or benzyl ester thereof or the sodium salt of the ester and other excipients such as dispersing agents and suspending agents.

The present invention also provides a method of preparing a pharmaceutical composition as hereinbefore described which comprises melting a saturated polyalkylene glycol glyceride, mixing a kinase inhibitor active ingredient with the molten glyceride and allowing the resulting mixture to solidify.

The glyceride is conveniently melted by heating to a temperature 10° to 20° C. above its melting point before addition of the kinase inhibitor active ingredient as a powder. Optional excipients may be added to the molten mixture.

When a composition of the invention is to be administered in capsules, for example orally, the liquid mixture of the kinase inhibitor active ingredient and glyceride may be poured into hard capsules or injected into soft capsules and allowed to solidify therein. Alternatively, the solid solution or solid dispersion obtained on cooling the liquid mixture of the kinase inhibitor active ingredient and glyceride may be remelted for introduction into capsules. The capsules may contain, for example, from 1 mg to 250 mg of the kinase inhibitor active ingredient.

When a composition of the invention is to be administered as a dispersion in an aqueous medium, e.g. water, a saline solution or mixture of water with a water-miscible pharmaceutically acceptable solvent, the solid solution or solid dispersion obtained on cooling the liquid mixture is conveniently broken up and dispersed in the aqueous medium by stirring or by ultrasonication.

Other suitable formulations for the administration of kinase inhibitors according to the methods of the present invention are set out in international patent application publication number WO00/48571.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2 to 7% mannitol, at a pH range of 4.5 to 5.5, that is combined with buffer prior to use.

After pharmaceutical compositions have been prepared, they can be placed in an appropriate container and labeled for treatment of an indicated condition. For administration of the kinase inhibitors disclosed herein, such labeling would include amount, frequency, and method of administration.

Pharmaceutical compositions suitable for use in the invention include compositions where the active ingredients are contained in an effective amount to achieve the intended purpose. The determination of an effective dose is well within the capability of those skilled in the art.

Various biodegradable and biocompatible polymeric matrices comprising the kinase inhibitors set out above, including microcapsules, nanospheres, and implants, are useful in the practice of the present invention.

Microspheres are fine spherical particles containing active drugs. They are differentiated from nanospheres primarily by the size of the particle; microspheres have a diameter of less than approximately 1000 μm, while nanospheres are submicronic (<1 μm). Microsphere systems contain either homogeneous monolithic microspheres, in which the drug is dissolved or dispersed homogeneously throughout the polymer matrix, or reservoir-type microspheres, in which the drug is surrounded by the polymer matrix membrane shell. Monolithic and reservoir systems can also be combined. For instance, active drug can be dispersed within, or adsorbed onto, the polymer surface in a reservoir-type microsphere.

Biodegradable polymers can consist of either natural or synthetic materials that vary in purity. Natural polymers include polypeptides and proteins (e.g., albumin, fibrinogen, gelatin, collagen), polysaccharides (e.g., hyaluronic acid, starch, chitosan), and virus envelopes and living cells (e.g., erythrocytes, fibroblasts, myoblasts). Natural materials require cross-linking in the microencapsulation process, leading to the denaturation of the polymer and the embedded drug. As a result, synthetic polymers are most commonly used. Frequently used synthetic polymers include poly(-hydroxy) acids such as polylactic acid (PLA), polyhydroxybutryic acid, and copoly (lactic/glycolic) acid (PLGA). These compounds are biocompatible, lack immunogenicity, and have physical properties that permit them to be easily shaped (to control the bioerosion rate).

Useful polymers include thermogels, i.e., hydrogels that alter their viscosity in response to changes in temperature. Such thermogels are known in the art and can contain, inter alia, an entangled network of two randomly grafted polymers, e.g., a network of poly(acrylic acid) and a triblock copolymer containing poly(propylene oxide) (“PPO”) and poly(ethylene oxide) (“PEO”) segments in the sequence PEO-PPO-PEO. This family of polymers goes by the trade name Pluronic polyols. Another Pluronic-based thermogel comprises Pluronic side chains grafted onto a bioadhesive backbone of either poly(acrylic acid) or chitosan. The thermogels useful in the invention are those that are liquid at room temperature, but that form gels at the normal temperature of the human body, i.e., about 37° C.

Colloidal particulate carriers can also be used in the methods of the present invention for delivering kinase inhibitor drugs. Liposomes are the preferred colloidal vehicle, and are composed of a phospholipid bilayer that may act as a carrier for both hydrophilic and hydrophobic medications. Liposomes can be made from, e.g., neutral lipids, charged phospholipids, and cholesterol. The addition of an amphophilic polymer such as polyethylene glycol (PEG) onto the surface of a liposome can slow the clearance of liposomes.

The disclosure of all patents, publications, (including published patent applications), and database accession numbers and depository accession numbers referenced in this specification are specifically incorporated herein by reference in their entirety to the same extent as if each such individual patent, publication, and database accession number, and depository accession number are specifically and individually indicated to be incorporated by reference.

The present invention is further illustrated with the following examples. However, the example is not to be construed as limiting the invention thereto.

### EXAMPLE 1

Intraperitoneally administered radiolabeled tracer can be used to quantitate BRB breakdown in mice exposed to vascular endothelial growth factor (VEGF). This technique is particularly advantageous with neonatal mice because of the technical difficulty involved with intravascular injections. Also, the comparison of retinas from injected and control eyes to tissues without a blood-tissue barrier corrects for variations in the amount of isotope injected or absorbed into the circulation.

Ten six- to seven-week-old C57B/J6 mice (Jackson Labs, Bar Harbor, Me.) are weighed to determine the proper dosage of anesthesia and tracer required. Mice are anesthetized with a solution containing 25 mg/kg ketamine (Ketaset; Fort Dodge Animal Health, Fort Dodge, Iowa) and 4 mg/kg rompun (Tranquived; Vedco, St. Joseph, Mo.) diluted 1:10 in saline prior to injection. Five mice are subjected to gavage with 50 mg/kg N-benzoyl staurosporine for three days prior to intravitreal administration of VEGF; the other five mice are subjected to gavage with vehicle.

Tropicamide (1%) is used to dilate the pupils of anesthetized mice. Under a dissecting microscope, a Harvard injector (PLI 100) is used with a glass pipet pulled to a diameter of 13-20 μm to inject 1 μl of saline containing VEGF (R&D Systems, Minneapolis, Minn.). The eyes are entered posterior to the limbus, directing the pipet toward the optic nerve. The solution is then deposited just in front of the retina without inflicting any damage to the lens. The eye are injected with VEGF at a concentration of 10⁻⁶ M. BRB integrity is assayed at 6 hours by administration of [³H]-mannitol tracer at 1 μCi/g. Sixty minutes after injection, the mice are injected with metofane and euthanized by cervical dislocation.

Retinas are rapidly removed by grasping behind the globe with Crile forceps and cutting across the anterior chamber with a Week razor blade. Retinas are isolated by quickly removing the contents of the posterior chamber with a curved Crile forcep, dissecting it free from RPE, lens, and vitreous, and placing it in a pre-weighed scintillation vial within 30 seconds of sacrifice. The thoracic cavity is opened at the xyphoid process, cutting through the sternum, and the left superior lobe of the lung is removed and placed in a pre-weighed glass vial, being careful not to nick the heart or pulmonary vein. The left kidney is removed dorsally and excess fat is trimmed away prior to placing it in another pre-weighed scintillation vial. The tissue is allowed to dry within the vials for 20 minutes, following which the vials containing tissue are weighed to determine the net weight of the tissue.

One ml NCSII (Amersham, Chicago, Ill.) solubilizing solution is added to each vial and the vials are incubated overnight in a 50° C. water bath. The solubilized tissue is brought to room temperature and decolorized with 20% benzoyl peroxide in toluene in a 50° C. water bath. The tissues are again brought to room temperature and 5 ml Cytoscint ES (ICN, Aurora, Ohio) and 30 μl glacial acetic acid is added. The vials are stored for several hours in darkness at 4° C. to decrease chemiluminescence. The specimens are counted on a Wallac 1409 Liquid Scintillation Counter (Gaithersburg, :Md.).

The counts per mg tissue are determined for treated (with N-benzoyl staurosporine) and untreated retina, lung, and kidney. Ratios of these values for treated or untreated retina/lung or kidney and for lung/kidney are calculated. The lung/kidney ratios obtained for retinas from animals treated with N-benzoyl staurosporine are compared to those gavaged with vehicle only using Student's t test with pooled variances.

It is found that the values of the retina/lung ratio (cpm per mg of retina/cpm per mg of lung) and the retina/kidney ratio are significantly reduced in the VEGF/N-benzoyl staurosporine-treated mice as compared to the VEGF/vehicle-treated mice. The retina/lung ratio in the VEGF/vehicle-treated mice is 0.8785±0.0946 versus 0.6326±0.0656 in the VEGF/N-benzoyl staurosporine-treated mice (Student-t value 0.0467). The retina/kidney ratio in the VEGF/vehicle-treated mice is 0.6806±0.1060 versus 0.3881±0.0436 in the VEGF/N-benzoyl staurosporine-treated mice (Student-t value 0.0201).

For comparison, control studies are done without administration of VEGF (i.e., no VEGF but administration by gavage of the vehicle for N-benzoyl staurosporine). The retina/lung ratio in the vehicle-only-treated mice is 0.422125±0.0771. The retina/kidney ratio in the vehicle-only-treated mice is 0.332125±0.0784. Both values are significantly different from those obtained from the VEGF and vehicle-treated mice.

Thus, there is significantly less radioactivity in the retinas of VEGF-treated mice treated with N-benzoyl staurosporine than in the retinas of untreated mice, indicating that there is significantly less leakage of radioactive tracer out of the retinal blood vessels of the N-benzoyl staurosporine-treated mice, demonstrating the utility of N-benzoyl staurosporine in decreasing leakage from retinal capillaries of the BRB in response to a known stimulator of such leakage, VEGF.

### EXAMPLE 2

Six to seven week old male C57BL/6J mice are treated in accordance with the ARVO resolution on the care of animals in research. Mice are treated by gavage with vehicle or vehicle containing 50 mg/kg/day of PKC412, PTK787, SU1498, or Gleevec. After 3 days of treatment, mice are given an intraocular injection of 1 μl of vehicle containing a vasopermeability factor or vehicle alone.

Mice are anesthetized with 25 mg/kg of ketamine and 4 mg/kg of xylazine, pupils are dilated with 1% tropicamide. Intraocular injections are performed as previously described with a Harvard pump microinjection apparatus and pulled glass micropipets. Each micropipet is calibrated to deliver 1 μl of vehicle upon depression of a foot switch. Under a dissecting microscope, the sharpened tip of a micropipet is passed through the sclera just behind the limbus into the vitreous cavity, and the foot switch is depressed. The following agents are tested: 10-6 M human vascular endothelial growth factor (VEGF; R&D Systems, Minneapolis, Minn.), 10-5 M human insulin-like growth factor-1 (IGF-1; R&D Systems), 10-5 M prostaglandin E1 (PGE1; Sigma, St. Louis, Mo.), 10-5 M prostaglandin E2 (PGE2; Sigma), 10-6 M human tumor necrosis factor-α (TNF-α; Sigma), and 100 units of human interleukin-1β (IL-1β; Chemicon, Temecula, Calif.). The left eye is injected with agent and the right eye is injected with vehicle or in some cases is left uninjected. Some mice are used for quantitative measurement of BRB breakdown using [3H]mannitol and in others leakage of albumin through retinal vessels is assessed by Western blots or immunohistochemistry.

Six or 24 hours after intraocular injections, mice are given an intraperitoneal injection of 1 μCi / gram body weight of [3H]mannitol (New England Nuclear, Boston, Mass.). After one hour, mice are sacrificed by cervical dislocation and eyes are removed. The cornea and lens are removed and the entire retina is carefully dissected from the eyecup and placed within pre-weighed scintillation vials. The thoracic cavity is opened and the left superior lobe of the lung is removed and placed in another pre-weighed scintillation vial. A left dorsal incision is made and the retroperitoneal space is entered without entering the peritoneal cavity. The renal vessels are clamped with a forceps and the left kidney is removed, cleaned of all fat, and placed into a pre-weighed scintillation vial. All liquid is removed from the vials and remaining droplets are allowed to evaporate over 20 minutes. The vials are weighed and the tissue weights are recorded. One ml of NCSII solubilizing solution (Amersham, Chicago, Ill.) is added to each vial and the vials are incubated overnight in a 50° C. water bath. The solubilized tissue is brought to room temperature and decolorized with 20% benzoyl peroxide in toluene in a 50° C. water bath. The vials are brought to room temperature and 5 ml of Cytoscint ES (ICN, Aurora, Ohio) and 30 μl of glacial acetic acid are added. The vials are stored for several hours in darkness at 4° C. to eliminate chemoluminescence. Radioactivity is counted with a Wallac 1409 Liquid Scintillation Counter (Gaithersburg, Md.).

Retinas are removed and homogenized in buffer containing 0.1% sodium dodecylsulfate in 0.1M Tris-HCl, pH 7.4. Lysates are centrifuged at 11,000×g for 10 minutes to remove large tissue debris. Samples are boiled and electrophoresed on NuPAGE 10% Bis-Tris gel (Invitrogen, Carlsbad, Calif.). After electrophoresis, gels are stained with Coomassie brilliant blue R-250. For immunoblotting, proteins are electrophoretically transferred from gels to ProBlott PVDF membranes (polyvinylidine difluoride; Applied Biosystems, Foster City, Calif.). After blocking with blocking solution (5% powdered nonfat dry-milk, 100 mM NaCl, 10 mM Tris-HCl, pH 7.4), the membranes are incubated for 1 hour with 1:10,000 rabbit anti-rat albumin (Nordic Immunological Labs, Tilburg, Netherlands) in blocking solution. The membranes are ished 4 times with ish solution (0.1% Triton X-100 in Tris-buffered saline) and incubated with 1:100,000 Horseradish peroxidase-conjugated anti-rabbit IgG antibody (Santa Cruz Biotechnology, Santa Cruz, Calif.) for 1 hour at room temperature. After washing 4 times with wash solution, the membrane is immersed in ECL Western blot reagent (Amersham, Piscataway, N.J.) and exposed to X-ray film (Eastman-Kodak, Rochester, N.Y.).

Immunohistochemical staining is done on 10 μm frozen sections of 4% paraformaldehyde-fixed eyes. Slides are incubated in methanol/H2O2 for 10 minutes at room temperature, washed with phosphate-buffered saline (PBS), pH 7.4, and blocked with 2% powdered nonfat dry milk in PBS for 30 minutes at room temperature in a humidified chamber. Excess milk solution is removed, and sections are covered with 1:500 rabbit anti-rat albumin in 2% powdered milk in PBS and incubated for 2 hours at room temperature. Sections are washed 3 times with PBS and incubated with 1:40 goat anti-rabbit IgG (Chemicon, Temecula, Calif.) in 2% powdered milk in PBS for 30 minutes. After 3 ishes, sections are incubated for 30 minutes with 1:100 peroxidase-antiperoxidase complex coupled to rabbit IgG (Chemicon). Slides are washed with PBS, incubated with diaminobenzidine (Research Genetics, Huntsville, Ala.) to give brown reaction product, and mounted with Cytoseal (Stephens Scientific, Riverdale, N.J.).

The CPM per mg tissue is measured for lung, kidney, treated retina, and untreated retina. Retina/lung, retina/kidney, and lung/kidney ratios are calculated. The ratios obtained for retinas treated with a particular agent at a specific concentration and time point are compared to those for untreated or saline-treated retinas using Student's unpaired t-test for populations with unequal variances.

It is found that blockade of protein kinase C by PKC412, but not VEGF receptor kinase by agents such as PTK787, reduces PG-induced breakdown of the BRB. Since inhibition of several isoforms of PKC is the only activity of PKC412 that is not shared by any of the other drugs tested, these data suggest that PGEs act through PKC to cause retinal vascular leakage. Thus, PKC412 can be beneficial for the treatment of macular edema in which prostaglandins play a role. Prostaglandins have been implicated in macular edema caused by intraocular surgery and ocular inflammatory disease, and prostaglandin synthetase inhibitors are widely used for treatment of these types of macular edema with limited success. PKC inhibitors alone, or addition of a PKC inhibitor to prostaglandin synthetase inhibitor(s) could be a substantial advance in the treatment of macular edema, since it should allow blockade at two sites in the molecular pathway leading to macular edema, and so can be useful in the treatment of multiple type of macular edema.

What is claimed is:

1. A method for decreasing or attenuating an increase in capillary permeability in the retina in a subject in need of such treatment, comprising administering a composition comprising an amount of a staurosporine derivative or a salt thereof to a subject suffering from excessive or pathological capillary permeability in the retina, the amount of staurosporine derivative or salt thereof being effective to decrease the permeability of capillaries in the retina of the subject.

2. The method of claim 1 wherein the subject is suffering from clinically significant macular edema.

3. The method of claim 1 wherein the subject is not suffering from clinically significant macular edema.

4. The method of claim 2 wherein the subject is suffering from visual acuity loss.

5. The method of claim 1, wherein the effective amount is less than about 150 milligrams per day.

6. The method of claim 5, wherein the effective amount is between about 50 and about 150 milligrams per day and the staurosporine derivative or a salt thereof is N-benzoyl staurosporine.

7. The method of claim 6, wherein the total effective amount is delivered to the subject in a single administration at a single time point during the day.

8. The method of claim 7, wherein the total effective amount is delivered to the subject in multiple administrations at multiple time points during the day.

9. The method of claim 2, wherein the effective amount is less than about 150 milligrams per day.

10. The method of claim 9, wherein the effective amount is between about 50 and about 150 milligrams per day.

11. The method of claim 10, wherein the total effective amount is delivered to the subject in a single administration at a single time point during the day.

12. The method of claim 11, wherein the total effective amount is delivered to the subject in multiple administrations at multiple time points during the day.

13. The method of claim 3, wherein the effective amount is less than about 150 milligrams per day.

14. The method of claim 13, wherein the effective amount is between about 50 and about 150 milligrams per day and the staurosporine derivative or a salt thereof is N-benzoyl staurosporine.

15. The method of claim 14, wherein the total effective amount is delivered to the subject in a single administration at a single time point during the day.

16. The method of claim 15, wherein the total effective amount is delivered to the subject in multiple administrations at multiple time points during the day.

17. The method of claim 4, wherein the effective amount is less than about 150 milligrams per day.

18. The method of claim 17, wherein the effective amount is between about 50 and about 150 milligrams per day and the staurosporine derivative or a salt thereof is N-benzoyl staurosporine.

19. The method of claim 18, wherein the total effective amount is delivered to the subject in a single administration at a single time point during the day.

20. The method of claim 19, wherein the total effective amount is delivered to the subject in multiple administrations at multiple time points during the day.

21. The method of claim 1, wherein the effective amount is administered topically to the eye.

22. The method of claim 2, wherein the effective amount is administered topically to the eye.

23. The method of claim 3, wherein the effective amount is administered topically to the eye.

24. The method of claim 4, wherein the effective amount is administered topically to the eye.

25. The method of claim 1, wherein the effective amount is administered intravitreally to the eye.

26. The method of claim 2, wherein the effective amount is administered intravitreally to the eye.

27. The method of claim 3, wherein the effective amount is administered intravitreally to the eye.

28. The method of claim 4, wherein the effective amount is administered intravitreally to the eye.

29. The method of claim 1 wherein the subject is not suffering from diabetes.

30. The method of claim 1, wherein the subject is suffering from a PGE-associated macular edema.

31. The method of claim 29, wherein the subject is suffering from macular edema wherein said macular edema is caused by a member selected from the group consisting of branch retinal vein occlusion, pseudophakic cystoid macular edema, aphakic cystoid macular edema, uveitis, idiopathic retinal telangiectasia, vitreomacular traction syndrome, dominantly inherited cystoid macular edema, and intraocular surgery.

32. A method for treating a subject suffering from diabetic nephropathy or diabetic neuropathy comprising administering an effective amount of a composition comprising an amount of a staurosporine derivative or a salt thereof to a subject suffering from diabetic nephropathy or diabetic neuropathy.

33. The method of claim 32, wherein the effective amount is less than about 150 milligrams per day.

34. The method of claim 33, wherein the effective amount is between about 50 and about 150 milligrams per day and the staurosporine derivative or a salt thereof is N-benzoyl staurosporine.

35. The method of claim 34, wherein the total effective amount is delivered to the subject in a single administration at a single time point during the day.

36. The method of claim 34, wherein the total effective amount is delivered to the subject in multiple administrations at multiple time points during the day.
